(19) United States
(12) Patent Application Publication
(10) Pub. No.: US 2006/0223078 A1
(43) Pub. Date: Oct. 5, 2006

(54) Method and device for detecting targets with a tape having probes capable of binding to the targets

(76) Inventors: Li Changming, Singapore (SG); Pan Likun, Singapore (SG)

Correspondence Address: DINSMORE & SHOHL LLP;One Dayton Centre, Suite 1300, One South Main Street, Dayton, OH, 45402-2023, US

(21) Appl. No.: 11/270,045

(22) Filed: Nov. 9, 2005

Related U.S. Application Data
(60) Provisional application No. 60/626,476, filed on Nov. 10, 2004.

(51) Int. Cl. C12Q 1/68; C12M 1/34; G01N 33/543

(52) U.S. Cl. 435/6; 435/287.2; 436/518

(57) ABSTRACT

A tape for detecting bio-analytes is provided. The tape includes a flexible, elongate band defining identifiable sections disposed lengthwise thereon. The tape also includes biological or biochemical probes. At least one probe is attached to a section. Probes in different sections are capable of binding specifically to different biological or biochemical targets. The tape can be enclosed in a cassette. Sections of the tape can be advanced sequentially to a test location. Whether a probe in a section has bonded to a corresponding target is detected when the section is at the test location. A detection system for use with the tape or cassette can include a mechanism for holding the tape and advancing the tape lengthwise through a detection location, and a detector disposed proximate the detection location, for detecting a signal originated from a section of the tape at the detection location.

### CROSS-REFERENCE TO RELATED APPLICATIONS

This application claims the benefit of and priority from United States provisional application No. 60/626,476, filed Nov. 10, 2004, the contents of which are incorporated herein by reference.

### FIELD OF THE INVENTION

The present invention relates to detection methods and devices.

### BACKGROUND OF THE INVENTION

Target molecules can be detected with probes disposed on a substrate, such as in a biochip. Each probe can be used to detect the presence of specific molecules at the probe. In some conventional techniques, the probes include electrodes and the target molecules are detected by measuring an electrical signal at each probe. The electrical signal detected at each probe varies depending on whether target molecules are present at the electrodes. An array of probe electrodes can be electrically addressable so that different target molecules may be detected at different probes simultaneously.

The conventional detection techniques have some drawbacks. One problem is that the conventional detection chips are not convenient to use in some applications. For example, it is difficult to form a large number of probes on a detection chip due to its limited size and the detection speed may be limited in large volume sample analysis. Another problem is that the addressing techniques used in the conventional detection methods and devices can be expensive to implement and inconvenient to use. Accordingly, there is a need for improved methods and devices of detection.

### SUMMARY OF THE INVENTION

Therefore, in an aspect of the present invention there is provided a tape. The tape includes a flexible, elongate band having a surface defining a plurality of identifiable sections disposed lengthwise thereon. The tape also includes a plurality of biological or biochemical probes, which may be immobilized at different sections of the tape. At least one of the probes is attached to each one of the sections. Probes in different sections are capable of binding specifically to different biological or biochemical targets. The binding in a particular section is detectable by a detector when the particular section passes by the detector.

In another aspect of the present invention there is provided a detection method. In this method, sections of a tape are advanced sequentially to a test location. The sections are disposed lengthwise on the tape and are identifiable. Each section includes a probe capable of binding with a specific target. Different probes are capable of binding specifically to different targets. Whether a probe in a section has bonded with a corresponding target is detected when the section is at the test location.

In a further aspect of the present invention there is provided a detection system. The detection system includes a mechanism for holding a tape and advancing the tape lengthwise through a detection location, and a detector disposed proximate the detection location, for detecting a signal originated from a section of the tape when the section is at the detection location. The signal is indicative of presence of a bonded structure formed from a probe attached to the section and a target.

In yet another aspect of the present invention there is provided a detection method. In this method, a plurality of test sites serially disposed on a flexible tape are sequentially advanced through a fixed detection position for detection. Each test site has attached thereto at least one probe capable of binding specifically to a target. Whether a specific target has bonded to a particular probe at a particular test site, such as after sample incubation, is detected when the particular test site is at the detection position. The particular probe is identified. The probe may be identified according to the serial position of the particular site on the tape.

In a further aspect of the present invention there is provided a cassette. The cassette includes a housing and a tape. The tape can be a tape described in any one of the four preceding paragraphs. The tape is housed in the housing such that the tape is advanceable along a path from a first end to a second end.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic view of a biotape;

FIG. 2 is a schematic partial view of a cassette system;

FIG. 3 is a schematic block diagram of a microfluidic system;

FIGS. 4A and 4B are respectively schematic partial views of another cassette system in two different operating positions;

FIG. 5 is a line graph of the imaginary part of measured impedance;

FIG. 6A to 6C are line graphs of measured capacitance changes as functions of target concentrations; and

FIG. 7 is a bar graph showing the dependence of measured capacitance changes on probe-target pairing.

### DETAILED DESCRIPTION

Briefly, a biosensing apparatus or method for electrical detection of biomolecules can be based on a cassette array tape platform. For example, the sensing components or probe molecules can be arrayed at different locations on the surface of a long mobile roll tape. When exposed to a complementary biomolecule target, the biosensing apparatus can produce a detectable change in an electrical or magnetic signal, or detect a radiation signal, to confirm the existence of a specific biomolecule target. A radiation signal can include an optical signal, an electromagnetic radiation signal, other radiation, and the like. Such a device or method can be used to rapidly and conveniently detect hundreds or thousands of different target molecules such as DNA molecules, RNA molecules, oligonucleotides, peptides, polypeptides, proteins, antibody, antigen, cells and the like.

As can be understood, the analyte in a particular application can be either the target or the probe, although it may be more convenient in some applications if the target is the analyte.

In general, an exemplary method according to an aspect of the present invention can include sequentially advancing a plurality of test sites serially disposed on a flexible tape through a fixed detection position for detection. Each test site can have attached thereto at least one probe capable of binding specifically to a target. Whether a specific target has bonded to a particular probe at a particular test site is detected when the particular test site is at the detection position. The particular probe is identified. The probe may be identified according to the serial position of the particular site on the tape.

Another exemplary embodiment of the present invention is a tape that has a flexible, elongate band. Identifiable sections are disposed lengthwise on a surface of the band. The band can comprise a material selected from plastic, rubber, fabric, insulated metal foils, soft or flexible printed circuit board (PCB), and the like. Each section can have attached thereto at least one biological or biochemical probe. Probes in different sections are capable of binding specifically to different biological or biochemical targets. The binding in a particular section is detectable by a detector when the particular section passes by the detector. The probes can be molecules immobilized on the tape, either covalently or non-covalently. The probes can include one or more of oligonucleotides, DNA molecules, RNA molecules, antibodies, antigens, peptides, proteins, and the like. A target can comprise one or more of oligonucleotides, DNA molecules, RNA molecules, antibodies, antigens, peptides, proteins, cells, and the like.

For electronic detection of the binding of target molecules to probe molecules, probe electrodes may be attached to sections of the tape for electrical coupling with an external electrode. When a probe is associated with a probe electrode, the binding of the probe with a target at the probe electrode can cause a detectable change in an electrical property, such as electrical capacitance. The change may be detected by applying an electrical signal to the probe electrode. The probe electrodes may comprise a material selected from gold, silver, platinum, copper, titanium, chromium, aluminum, metal oxide, metal carbide, carbon, graphite, fullerene, conductive plastic, conductive polymer, metal impregnated polymer, and the like.

The probe electrodes may form one or more arrays aligned lengthwise. The tape may have a conducting line for each array, sequentially connecting the probe electrodes in the array. Thus, electrodes in each array are individually identifiable depending on their lengthwise positions on the tape. When more than one arrays are provided, probe electrodes in different arrays can be electrically isolated such that electrodes in different arrays are separately identifiable.

The tape may also be magnetic and the binding in a particular section may be detectable by measuring a magnetic property of the particular section.

The binding in a particular section may alternatively be detectable by detecting a radiation signal, which is emitted from the particular section and is indicative of the binding in the particular section. A radiation signal can include an optical signal, an electromagnetic signal, other radiation signals, or the like.

The tape may also have a detectable marker attached to each section for identifying that section.

For convenience of use, the tape may be enclosed in a cassette, exemplary of embodiments of the present invention. The cassette includes a housing and the tape. The tape is housed in the housing such that the tape is advanceable along a path from a first end to a second end. The cassette may have a sampling chamber disposed along the path for receiving a sample such that a portion of the tape is contacted by the sample when the portion is advanced from the first end to the second end. The cassette may also have a washing chamber disposed along the path for receiving a washing solution, the washing solution capable of removing one or more of unbonded and non-specifically bonded targets from the tape. The cassette may further have a drying chamber disposed along the path for removing water from the tape. The sampling chamber, washing chamber and drying chamber can be integrated into a single chamber.

In use, sections of the tape are advanced sequentially to a test location. Whether a target has bonded to a corresponding probe in a section is detected when the section is at the test location.

The detection operation can be performed using a detection system, exemplary of embodiments of the present invention. The detection system includes a mechanism for holding a tape and advancing the tape lengthwise through a detection location, and a detector disposed proximate the detection location, for detecting a signal originated from a section of the tape when the section is at the detection location. The signal is indicative of the presence of a bonded structure formed from a probe attached to the section and a target.

The detection system may have reels for carrying the tape and a motor for driving the reels. It may have a circuitry for controlling rotation of the reels and for identifying the section at the detection location. The detection system may have a computer processor operatively connected to the detector for processing signals detected from the detection location. The detector may have an electrode for coupling with an electrode disposed on the tape to detect electronic signals.

Exemplary embodiments of the present invention are further illustrated in more details with reference to the figures.

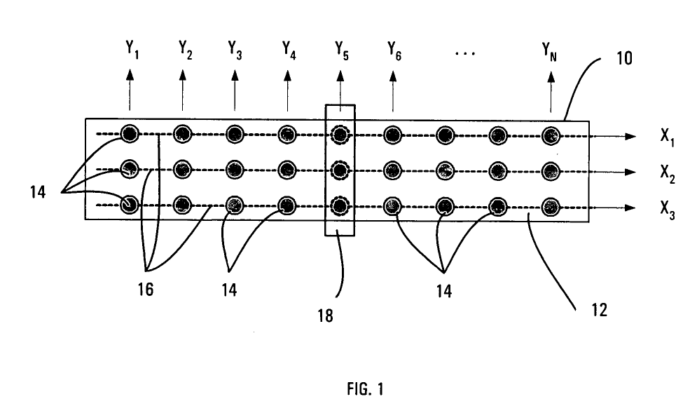

FIG. 1 illustrates a biotape 10, exemplary of embodiments of the present invention. Biotape 10 provides an addressable array platform on substrate 12, which may be flexible. An array of test sites 14 are formed on substrate 12 in rows, denoted as Xᵢ (i=1, 2, . . . , m), and columns, denoted as Yⱼ (j=1, 2, . . . , n). Each test site 14 can be identified by its row and column indices, such as TS_{i,j} for the test site at row “i” and column “j”.

For electronic detection of the binding of target molecules to the probe molecules at the test sites 14, each test site can include an electrode formed on substrate 12. The electrodes thus form an x-y addressable array. Each electrode can be identified by its row and column indices, such as E_{i,j} for the electrode at row “i” and column “j”. The electrodes in each row are electronically interconnected, such as by a conducting wire 16. The electrodes in each column are not electronically interconnected.

Substrate 12 of biotape 10 may be made of any suitable material. In some applications, the material should be sufficiently strong, smooth, and flexible so that it is suitable for making tapes. The material may also be limp. The material can be selected from, but not limited to, plastic, rubber, fabric, insulated metal foils, soft printed circuit board, or combinations thereof. Biotape 10 can be substantially flat as in a normal tape. For example, substrate 12 can be formed from a conventional plastic or metal tape used in audio recording applications, such as in audio cassettes. Further, substrate 12 can be made of commercially available and inexpensive plastic films, such as polyethylene, polyimide, Teflon, and the like.

Electrodes at test sites 14 may be made of any suitable material. The material may be selected from, but not limited to, gold, silver, platinum, copper, titanium, chromium, aluminum, metal oxide, metal carbide, carbon, graphite, fullerene, conductive plastic, conductive polymer, metal impregnated polymers, or combinations thereof.

For forming electrodes at test sites 14 on biotape 10, such as in an addressable micrometer-scale array, suitable techniques such as screen printing or inkjet printing methods can be used. For forming nanoscale electrodes on biotape 10, soft lithography or Dip-Pen Nanolithography (DPN) based Atomic Force Microscopy (AFM) technology can be used. For further development, Zerox™ machines can be developed to print patterned array electrodes on the tape.

Different probe molecules (not shown) may be immobilized at test sites 14 for detecting different target biomolecules.

Probes for different applications such as the detection of HIV, breast cancer, or liver cancer can be formed on the same biotape. Thus, a flexible detection system can be designed for detecting a variety of different targets.

The probe molecules may be immobilized on test sites 14 within a matrix. The probe molecules can be either covalently or non-covalently immobilized. The probe molecules may include one or more of oligonucleotides, DNA molecules, RNA molecules, proteins, peptides, antibodies, antigens, other small molecules, and the like. The probes attached to tape 10 may also include any other suitable biological or biochemical probes, such as those used in biological or biochemical analysis or analyte detection. The probe molecules or biomarkers can be immobilized on different spots (different test sites 14) with existing matured nanodispensor technology for the μ-array biotapes, or with DPN AFM technology for the nanoarray biotapes.

In different embodiments, the biotape can also be a magnetic biotape, which can be used to magnetically detect the biomolecular interactions by either labeling or labelless detection schemes. The test sites on the magnetic biotape can be similarly arranged as on biotape 10.

In different embodiments, the biomolecular interactions may be optically detected by labeling detection schemes. The test sites on the biotape can be similarly arranged as on biotape 10.

Further, in some embodiments, identification markers may be attached to the tape wherein each marker is associated with a probe for identifying the probe. For example, the electrodes in row X₃ in FIG. 1 may be used to read identification data embedded on the tape for identifying the types of probes in each column.

A biotape according to embodiments of the present invention can be fabricated using low cost mass production process or techniques for producing music tapes, which use a reel-to-reel mechanism, with some suitable modifications, as will be understood by persons skilled in the art. For instance, a difference between producing a video/radio tape and a tape for bio-detection is that in the production of the latter, a conventional biochip manufacturing process can be used to attach different probe molecules onto different array electrode spots on the tape, for example, through a robot nanodispenser machine. The biotape can contain many different biochips made by existing technologies.

During use, a reader 18 can be used to detect the binding of target molecules to the immobilized probes at the test sites 14. Reader 18 can have a counter electrode such as a microband electrode. As depicted in FIG. 1, reader 18 can be aligned along the y-direction above a column of electrodes at test sites 14, such as Y₅, so that electrical signals can be detected from electrodes in the column.

The detected electrical signals may indicate the presence or absence of target molecules at each test site 14. For example, after binding of probe and target molecules, the change in capacitance between reader 18 and a particular addressed electrode, such as E_{1,5}, due to the change of charge and dielectric coefficient can be labellessly and electronically detected.

A digital logic comparator (not shown) can be provided to produce a signal representative of whether the target molecule is bonded or not, in response to the capacitance change.

Biotape 10 can be advanced longitudinally or lengthwise to pass by reader 18 so that each column of addressed test sites 14 are read sequentially. For example, biotape 10 may be wound to move different columns of test sites 14 under reader 18 for detection.

As can be understood, biotape 10 can have very high density of test sites 14 and much less I/O lines than in conventional addressable electronic bioarray chips, since no I/O lines are necessary for addressing the electrode columns (Y) because they can be sequentially, and automatically, identified when moved by reader 18. Only electrode rows (X) may require electronic addressing for multiplexing detection.

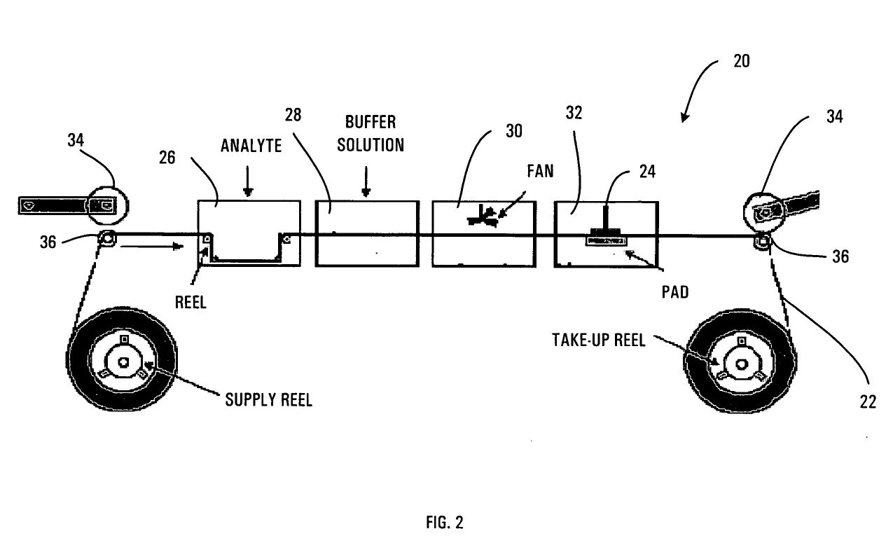

FIG. 2 is a schematic view of a cassette biotape system 20, exemplary of embodiments of the present invention.

System 20 has a tape guiding system for advancing a tape 22, which can be similarly formed as biotape 10, to pass by a reader 24, which may be an electrode for electronic detection of the binding of target molecules to probe molecules at the test sites. A microfluidic system may be used to deliver and process samples to be detected. In an example microfluidic system as depicted in FIG. 2, there are four major chambers for different functions, incubation chamber 26, washing chamber 28, drying chamber 30, and detection chamber 32. The tape guiding system includes pinch rollers 34 and capstans 36.

The baselines for each test site on tape 22 can be tested after probe molecules are immobilized and stored in cassette biotape system 20.

In use, the sample to be tested is delivered to the micro incubation chamber 26. As shown, a selected section of tape 22 containing the required probe molecules is moved into incubation chamber 26 for incubation. After incubation, the section of biotape travels into washing chamber 28 for washing the unbonded or nonspecific bonded biomolecules away for specificity. Then the section of tape goes into drying chamber 30 to remove water by air flow. The last step is sending the section of tape into detection chamber 32, to allow reader 24 to read, for example, the capacitance for electronic detection of the binding of probe-target molecules.

The measured results at the test sites are compared to the pre-stored baselines to determine whether a molecular interaction between the probe and the target molecule has occurred for further confirmation whether the target molecule is present in the sample mixture tested.

In this exemplary embodiment, the principle of a cassette tape recorder has been employed. In system 20, tape 22 can move at a constant rate. For example, the tape can be pulled across a tape head when each pinch roller 34 pinches tape 22 between itself and a corresponding capstan 36. One or both capstans 36, can be driven by a motor (not shown), for advancing tape 22 at a selected speed.

The chambers 26, 28, 30 and 32 can include buffer and sample transportation channels, which can be fabricated as a micro-electro-mechanical system (MEMS). MEMS technology can provide microchambers for use in system 20, as well as microchannels, micropumps and microvalves for transporting solutions. However, as can be appreciated, for a biotape cassette sized like a regular music tape cassette, it is not necessary to use MEMS technology.

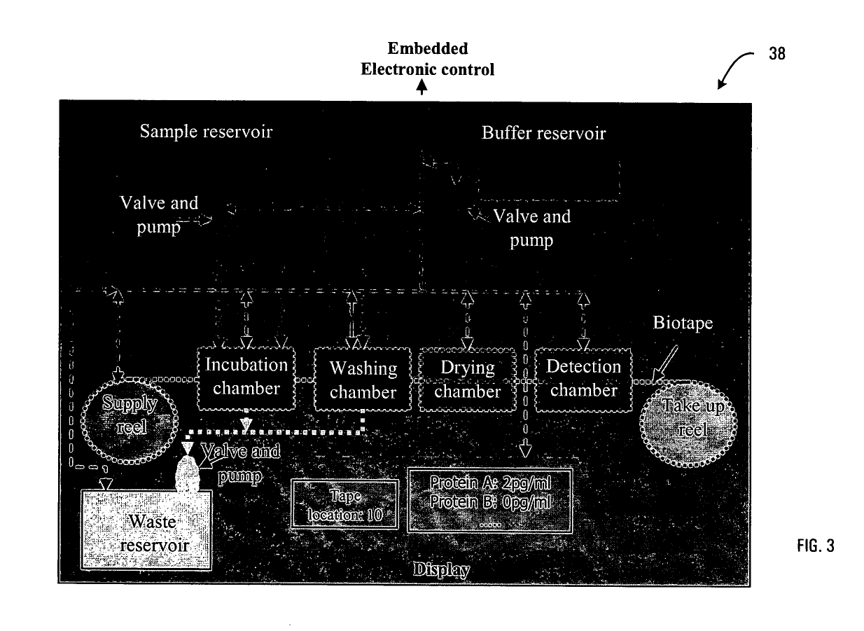

FIG. 3 is a schematic block diagram illustrating an example microfluidic system 38 similar to system 20. System 38 is a MEMS based sample handling system and includes miniaturized valves, pumps and fluid tubing such as plastic tubing (indicated by dashed lines). As illustrated, system 38 can be controlled with an embedded electronic control system, and the operation status and results can be displayed on a display to a user.

In alternative embodiments, a biotape cassette can have a single incubation chamber for conducting the four operations describe above.

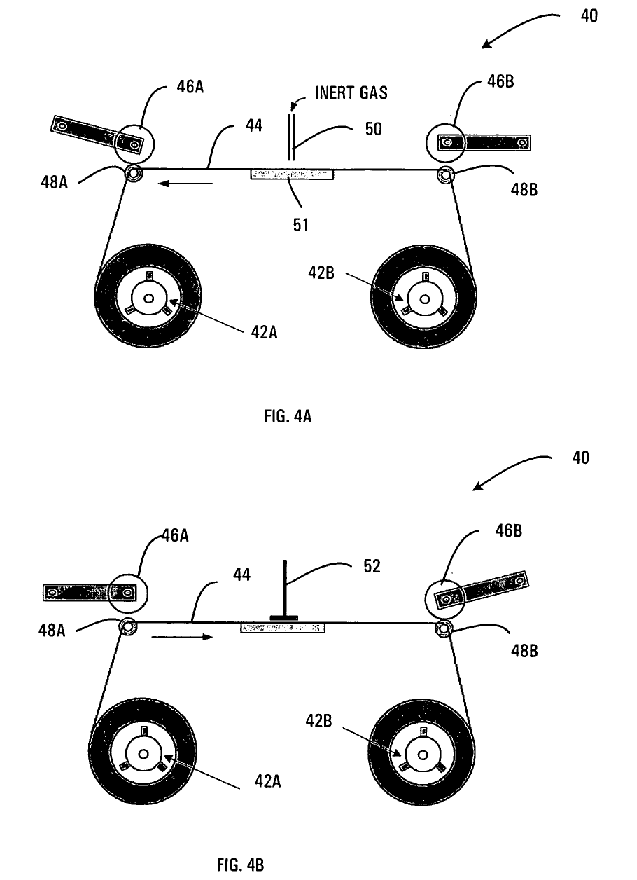

A further cassette system 40, exemplary of embodiments of the present invention, is schematically illustrated in FIGS. 4A and 4B.

As shown in FIG. 4A, system 40 has a reel 42A, which can spool the tape 44 onto reel 42A for take-up after it passes through the pinch roller 46A and capstan 48A.

As shown in FIG. 4B, when the direction of tape travel is reversed, the previously active pinch roller 46A can be pulled away from capstan 48A, allowing tape 44 to pass freely by capstan 48A. Pinch roller 46B, which was previously not active, is now pushed against capstan 48B. Reel 42B now becomes the take-up reel.

As can be appreciated, each of capstans 48A and 48B can rotate in both directions. The direction of tape travel can change in different procedures when reels 42A and 42B switch between functioning as supply or take-up reels and pinch rollers 46A and 46B respectively move to abut one of capstans 48A and 48B, and away from the other capstan.

System 40 has a number of functional heads which can be alternatively positioned as the tape head against tape 44. The possible functional heads may include a reader, for example an electrode for electronic detection, a dispenser, a pipettor, and a gas gun. System 40 can thus perform impedance measurement, solution dispensing, washing, and drying functions by switching to different functional heads. Alternatively, system 40 may have a multifunctional tape head. As can be appreciated, a traditional magnetic head for a conventional audio tape may not be suitable for use in system 40.

As can be understood, by changing the direction of tape travel, different operations or procedures can be performed in a single chamber. For example, as depicted in FIG. 4A, a drying head 50 may be positioned as the tape head, opposite a pad 51, for drying a section of tape 44 after incubation and washing, when sections of tape 44 are advanced towards the left-hand side. As depicted in FIG. 4B, a reader 52 may be positioned as the tape head for detection operation, when sections of tape 44 are advanced towards the right-hand side.

Advantageously, an embodiment of the present invention may provide one or more of the following benefits. A cassette biotape system using addressable array electrodes as described herein can significantly reduce I/O lines, even in comparison to known electronic x-y addressing methods used in current biochips, by one half. This will significantly reduce the cost of detection instrumentation and tape cost. The electrode or detection site density on a tape according to embodiments of the present invention can be much higher than that on a conventional bioarray chip. It is possible to perform labelless detection using embodiments of the present invention, such as impedance and magnetic measurement with high throughout for highly sensitive detection. The tapes and detection systems can be manufacture at low cost. Mass production manufacturing process is possible for producing the tapes. The operation of the exemplary cassette system and tapes can be simple and automatic, and thus significantly reduces total assay time. The tapes and detection systems can be miniaturized, such as for easy storage. One embodiment of the present invention can be used to conduct different electronic, magnetic, electromagnetic, optical, or radiation detections.

As can be appreciated, the exemplary methods of detection disclosed herein can be simple and sensitive. A cassette array biotape recorder can be used as a mobile electrical detection device for biomolecule detection without the need for labeling. It is also within the scope of the present invention to fabricate a high density biosensor with addressable electrodes coated with thousands of sensing probes for screening applications. In comparison with conventional biochip technology, a biotape system can provide higher array density (without any theoretical limitation), and more applications can be performed on a single tape. Further, a tape within the scope of this invention can be operated similarly as a regular audio tape. Detected information can be recorded or retained on the tape for later use. It is convenient to automatically choose a particular application with a given tape section, such as an application for HIV detection, liver cancer detection, and the like, in terms of requirements. A unique and universal platform can be provided with embodiments of the present invention. Embodiments of the present invention can be powerful tools in many important genome and proteome applications. They can potentially replace conventional biochip platforms for many applications.

Potential application areas for the present invention include diagnostics, therapeutics, pre-clinical and clinical trials, target discovery, target validation, pathogen detection for drug discovery, health care, food processing, environmental monitoring, defense industry, and the like.

For example, embodiments of this invention can be used to make an innovative protein biosensor for immunoassay. This can provide high throughput and a sensitive method for clinical laboratory diagnosis. The combination of these features with miniaturization, low cost, and essentially real-time measurements in a variety of applications can be of significant commercial value.

The present invention is further illustrated by the following non-limiting examples.

### EXAMPLE II

A prototype biotape was made with a plastic tape. Addressable gold array electrodes at each test site on the tapes were formed with a PCB manufacturing process. The prototype system was operated and it was found to be functional.

Gold array electrodes were deposited on the surface of the prototype tape. The electrodes were immersed in a 1 mM 11-Mercapto-undecanoic acid (linker) solution in absolute ethanol for 6 hours and then vigorously rinsed in absolute ethanol. The electrodes were then immersed in a phosphate buffer solution (PBS) containing 100 ug/ml rabbit IgG, as probes. After 5 hours of incubation, the electrodes were vigorously rinsed in a PBS solution and dried. After the probe molecules were deposited on the gold electrodes surface, a Solartron Impedance Frequency Analyzer 1260 with Impedance Interface 1294 was used to measure the impedance of each array electrode in air. A two-electrode system was constructed. Sample solutions of 0, 10 pg/mi, 100 pg/ml, 1 ng/ml, 10 ng/ml, 100 ng/ml, 1 ug/ml, 10 ug/ml, and 100 ug/ml anti rabbit IgG in PBS solutions were prepared. After baseline reading, the array electrodes were incubated in each of these solutions for 2 hours, respectively. The electrodes were then rinsed vigorously in a PBS solution and dried. Impedance measurements were taken at each electrode again.

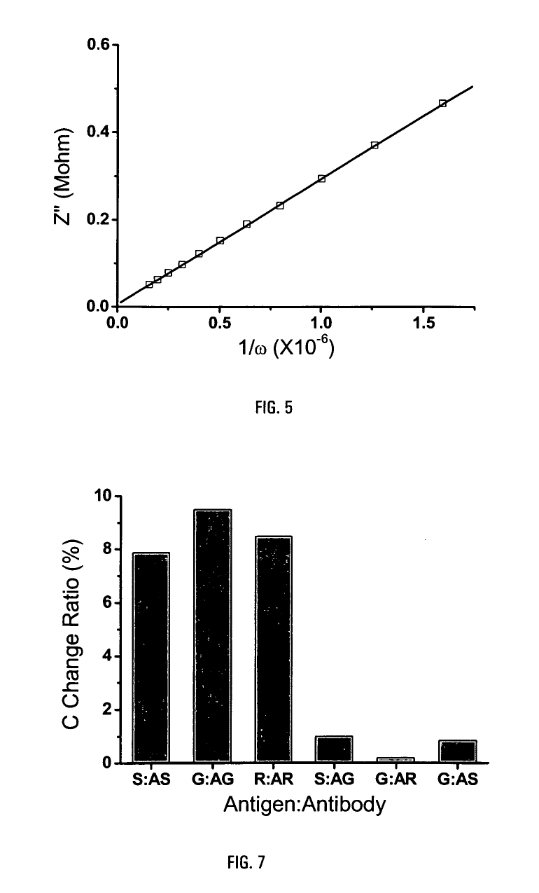

It is known that the imaginary part of impedance is in a reverse proportion to the frequency for pure capacitor: ZΔ=1/(ωC). As a result, the capacitance C can be calculated by the slope of the straight line determined by the variation of ZΔ as a function of 1/ω. FIG. 5 shows an example line graph of the measured imaginary part of impedance as a function of 1/(ω). The change in capacitance for each electrode before and after incubation was obtained from the slope of the line.

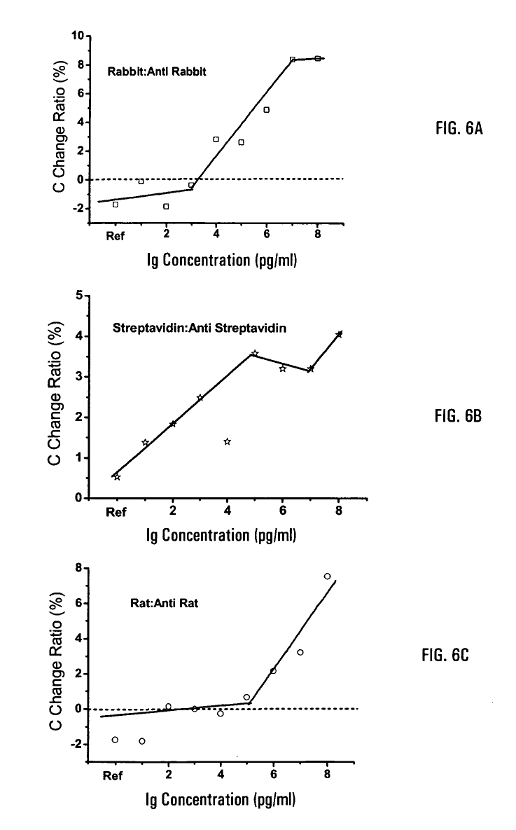

Calculated ratio of capacitance change is shown in FIG. 6A as a function of Ig concentration. The average of change in capacitance for each of the solutions is plotted versus the concentration of each of the solutions. The PBS solution without anti rabbit IgG is used as the reference. These results demonstrate that the presence of target analyte can be detected using embodiments of the present invention, at a sensitivity down to at least 10 ng/ml, with a dynamic range of over three-orders of magnitude to reach a plateau response.

### EXAMPLE II

100 ug/mL rat IgG as probe molecules in a PBS solution were immobilized on the gold array electrodes by linkers using the same procedure described in Example I. Before the samples were detected, AC impedance at the array electrodes was measured to obtain a baseline reading. The array electrodes were incubated in solutions of 0, 10 pg/ml, 100 pg/ml, 1 ng/ml, 10 ng/ml, 100 ng/ml, 1 ug/ml, 10 ug/ml, and 100 ug/ml anti rabbit IgG for 2 hours, respectively. The electrodes were rinsed vigorously in a PBS solution and dried. Impedance measurements were taken at each electrode. The average capacitance change for each of the solutions is plotted versus the concentration of each of the solutions in FIG. 6B. These results further demonstrate the capacitance change due to antibody binding.

### EXAMPLE III

100 ug/mL streptavidin as probe molecules in a PBS solution were immobilized on the gold array electrodes by linkers using the procedure described in Example I. After a baseline reading, the electrodes were incubated in solutions of 0, 10 pg/ml, 100 pg/ml, 1 ng/ml, 10 ng/ml, 100 ng/ml, 1 ug/ml, 10 ug/ml, and 100 ug/ml anti streptavidin for 2 hours, respectively. The electrodes were vigorously rinsed by PBS solution and dried. AC impedance measurements were conducted again and plotted in FIG. 6C. These results further confirm that this electrical detection method can be used to detect biomolecule targets.

### Example IV

100 ug/ml rabbit IgG, goat IgG, and streptavidin as probe molecules in PBS solution had been immobilized respectively on the gold array electrodes by the linker using the procedure described in Example I. After a baseline reading, the electrodes were incubated in 100 ug/ml different or corresponding antibody solutions for 2 hours, respectively. FIG. 7 shows the ratio of capacitance change for each electrode, plotted versus different antigen and antibody pairs, where S=streptavidin; AS=anti streptavidin; G=goat IgG; AG=anti goat IgG; R=rabbit IgG; AR=anti rabbit IgG. These results show the cross-reaction rate of different antibody target to antigen probe is low, demonstrating excellent specificity of this labelless detection technology.

Other features, benefits and advantages of the embodiments described herein not expressly mentioned above can be understood from this description and the drawings by those skilled in the art.

Of course, the above described embodiments are intended to be illustrative only and in no way limiting. The described embodiments are susceptible to many modifications of form, arrangement of parts, details and order of operation. The invention, rather, is intended to encompass all such modification within its scope, as defined by the claims.

What is claimed is:

1. A tape comprising:
a flexible, elongate band having a surface defining a plurality of identifiable sections disposed lengthwise thereon;
a plurality of biological or biochemical probes, at least one of said probes being attached to each one of said sections, said probes in different sections capable of binding specifically to different biological or biochemical targets, said binding in a particular section detectable by a detector when said particular section passes by said detector.

2. The tape of claim 1, further comprising a plurality of probe electrodes attached to said tape for electrical coupling with an external electrode, each one of said probes associated with one of said probe electrodes, and wherein the binding of each one of said probes with its corresponding target causes a change in an electrical property detectable when an electrical signal is applied to its associated probe electrode.

3. The tape of claim 2, wherein said probe electrodes form at least one array aligned lengthwise, and said tape further comprising a conducting line for each one of said at least one array, sequentially connecting said probe electrodes in said each array, such that said electrodes in said each array are individually identifiable depending on their lengthwise positions on said tape.

4. The tape of claim 3, wherein said at least one array comprise a plurality of arrays, said probe electrodes in different arrays being electrically isolated such that said electrodes in different arrays are separately identifiable.

5. The tape of claim 1, wherein said probes are molecules immobilized on said tape, either covalently or non-covalently.

6. The tape of claim 5, wherein said probes comprise one or more of oligonucleotides, DNA molecules, RNA molecules, antibodies, antigens, peptides, and proteins.

7. The tape of claim 6, wherein each one of one or more of said targets comprises one or more of oligonucleotides, DNA molecules, RNA molecules, antibodies, antigens, peptides, proteins, and cells.

8. The tape of claim 1, wherein said band comprises a material selected from plastic, rubber, fabric, insulated metal foil, and flexible printed circuit board.

9. The tape of claim 2, wherein said electrical property is electrical capacitance.

10. The tape of claim 2, wherein said probe electrodes comprise a material selected from gold, silver, platinum, copper, titanium, chromium, aluminum, metal oxide, metal carbide, carbon, graphite, fullerene, conductive plastic, conductive polymer, and metal impregnated polymer.

11. The tape of claim 1, wherein said tape is magnetic and said binding in a particular one of said section is detectable by measuring a magnetic property of said particular section.

12. The tape of claim 1, wherein said binding in said particular section is detectable by detecting a radiation signal indicative of said binding emitted from said particular section.

13. The tape of claim 12, wherein said radiation signal comprises an optical signal or an electromagnetic signal.

14. The tape of claim 1, further comprising a detectable marker attached to each one of said sections for identifying said each section.

15. A detection method comprising:
advancing sections of a tape sequentially to a test location, said sections being disposed lengthwise on said tape and identifiable, each one of said sections including a probe capable of binding with a specific target, different ones of said probes capable of binding specifically to different targets; and
detecting, when each one of said sections is at said test location, whether a probe in said each section has bonded to a corresponding target.

16. The method of claim 15, wherein said tape has a plurality of probe electrodes attached thereto for electrical coupling with an external electrode, each one of said probes associated with one of said probe electrodes, and wherein said detecting comprises applying an electrical signal to a probe electrode associated with said probe in said each section and sensing an electrical response indicative of whether said probe in said each section has bonded to said corresponding target.

17. The method of claim 16, wherein said probe electrodes form at least one array aligned lengthwise, and said method further comprising identifying a particular one of said probe electrodes based on its order or time of arrival at said test location.

18. The method of claim 17, wherein said at least one array comprise a plurality of arrays disposed laterally on said tape, said probe electrodes in different arrays being electrically isolated, and wherein said method further comprises identifying each one of said electrodes in a particular one of said arrays based on the lateral position of said particular array on said tape.

19. The method of claim 16, wherein said probe electrodes form at least one array aligned lengthwise, and said method further comprising identifying a particular one of said sections by detecting a marker attached to said section which identifies said section.

20. The method of claim 16, wherein said electrical response is indicative of an electrical capacitance.

21. The method of claim 15, wherein each one of one or more of said targets comprises one or more of oligonucleotides, DNA molecules, RNA molecules, antibodies, antigens, peptides, proteins, and cells.

22. The method of claim 15, wherein said probes comprise one or more of oligonucleotides, DNA molecules, RNA molecules, antibodies, antigens, peptides, and proteins.

23. A detection system, comprising:
a mechanism for holding a tape and advancing said tape lengthwise through a detection location; and
a detector disposed proximate said detection location, for detecting a signal originated from a section of said tape when said section is at said detection location, said signal indicative of presence of a bonded structure formed from a probe attached to said section and a target.

24. The system of claim 23, wherein said target comprises one or more of oligonucleotides, DNA molecules, RNA molecules, antibodies, antigens, peptides, proteins, and cells.

25. The system of claim 23, wherein said probe comprises one or more of oligonucleotides, DNA molecules, RNA molecules, antibodies, antigens, peptides, and proteins.

26. The system of claim 23, further comprising said tape held by said mechanism for holding.

27. The system of claim 23, wherein said mechanism comprises a first reel for holding a roll of said tape mounted before said detection location and a second reel for holding a roll of said tape mounted after said detection location.

28. The system of claim 27, further comprising a motor for rotating one or more of said reels so as to advance said tape.

29. The system of claim 28, further comprising a circuitry for controlling said rotating and for identifying said section at said detection location.

30. The system of claim 29, further comprising a sampling chamber for contacting said probe with a sample solution.

31. The system of claim 30, further comprising a washing chamber for removing one or both of unbonded and non-specifically bonded targets from said tape.

32. The system of claim 31, further comprising a drying chamber for removing water from said tape.

33. The system of claim 32, where said sampling chamber, washing chamber and drying chamber are integrated into a single chamber.

34. The system of claim 23, further comprising a processor operatively connected to said detector for processing said signal.

35. The system of claim 23, wherein said signal comprises at least one of electronic, magnetic, electromagnetic, optical, and radiation signals.

36. The system of claim 23, wherein said detector comprises an electrode for coupling with an electrode disposed on said tape.

37. The system of claim 28, further comprising one or more pinch rollers for controlling advancing of said tape.

38. A detection method comprising:
sequentially advancing a plurality of test sites serially disposed on a flexible tape through a fixed detection position for detection, each one of said test sites having attached thereto at least one probe capable of binding specifically to a target;
detecting, when a particular one of said test sites is at said detection position, whether a specific target has bonded to a particular probe at said particular test site; and
identifying said particular probe.

39. The method of claim 38, wherein said identifying comprising identifying said particular probe according to the serial position of said particular site on said tape.

40. A cassette comprising a housing and the tape of claim 1 housed in said housing such that said tape is advanceable along a path from a first end to a second end.

41. The cassette of claim 40, further comprising a sampling chamber disposed along said path for receiving a sample such that a portion of said tape is contacted by said sample when said portion is advanced from said first end to said second end.

42. The cassette of claim 41, further comprising a washing chamber disposed along said path for receiving a washing solution, said washing solution capable of removing one or both of unbonded and non-specifically bonded targets from said tape.

43. The cassette of claim 42, further comprising a drying chamber disposed along said path for removing water from said tape.

44. The cassette of claim 43, wherein said sampling chamber, said washing chamber and said drying chamber are integrated into a single chamber.
